(19) United States
(12) Patent Application Publication
(10) Pub. No.: US 2026/0297655 A1
(43) Pub. Date: Oct. 1, 2026

(54) METHOD FOR DETECTING A SUBSTANCE OF INTEREST USING NUCLEIC ACID APTAMER BEACONS

(71) Applicant: HONDA MOTOR CO., LTD., TOKYO (JP)

(72) Inventors: Tatsuo NAKAMURA, YOKOHAMA-SHI (JP); Yuko NAKAMURA, YOKOHAMA-SHI (JP); Tadahisa NAKAMURA, WAKO-SHI (JP); Tsuyumi NISHIYAMA, WAKO-SHI (JP); Toshiaki SUMI, WAKO-SHI (JP)

(21) Appl. No.: 19/574,916

(22) Filed: Mar. 23, 2026

(30) Foreign Application Priority Data
Mar. 27, 2025 (JP) 2025-054284

(51) Int. Cl. C12Q 1/6818; C12Q 1/682; C12Q 1/6825

(52) U.S. Cl. CPC C12Q 1/6818; C12Q 1/682; C12Q 1/6825

(57) ABSTRACT

A method to the sensitivity of nucleic acid aptamer beacons, which in one respect involves detecting a target substance using a nucleic acid aptamer beacon. The wherein the method includes: (A) a step of mixing a target substance, a nucleic acid aptamer beacon, and a response-enhancing nucleic acid; and (B) a step of measuring light emission from the mixture, wherein the response-enhancing nucleic acid includes a region hybridizable to a portion of at least one of the region of the first nucleic acid and the region of the second nucleic acid of the nucleic acid aptamer.

### INCORPORATION BY REFERENCE

The material contained in the Sequence Listing XML file named “AIF-101-A_Sequence-Listing-revised”, created on Mar. 23, 2026, and having a file size of 4677 bytes, is hereby incorporated by reference in its entirety into the present application.

### FIELD OF THE INVENTION

This disclosure relates to a method for detecting a substance of interest (a target substance) using a nucleic acid aptamer beacon.

### BACKGROUND OF THE INVENTION

Various analytical methods are used for the purpose of detecting a target substance. One such analytical method involves the use of nucleic acid aptamers. Nucleic acid aptamers have nucleic acid sequences that can specifically bind to a target substance. Upon binding to the target substance, a conformational change is caused. The nucleic acid aptamer beacon takes advantage of such a conformational change. Specifically, the nucleic acid aptamer beacon alters the degree of light emission in a conformational change-dependent manner.

Patent Literature 1 discloses a mechanism that causes a change in the degree of light emission. Specifically, Patent Literature 1 discloses a first reporter group and a second reporter group, wherein the first reporter group is a fluorophore and the second reporter group is a chemical quencher. When a conformational change occurs, the distance between the two reporter groups changes, thereby causing a change in the degree of light emission.

### CITATION LIST

### Patent Literature

[Patent Literature 1] WO 00/070329 A1 (Japanese Patent Application Publication No. 2003-508729 A)

### SUMMARY OF THE INVENTION

Nucleic acid aptamer beacons are typically produced by the following procedure:
screening nucleic acids based on the interaction between the target substance and the nucleic acid;
isolating candidate nucleic acids based on the screening results; and
producing a nucleic acid aptamer beacon from the isolated candidate nucleic acid, which functions or is likely to function as a nucleic acid aptamer.

In reality, however, in some cases, nucleic acid aptamers that exhibit performance sufficient to achieve the intended purpose may not be obtained. For example, in some cases, the nucleic acid aptamer binds to the target substance but may not be sufficiently sensitive as a nucleic acid aptamer beacon.

In such cases, searching again for candidate nucleic acids with different sequences requires a great deal of effort.

For these reasons, an object of the present disclosure is to provide means for enhancing the sensitivity of nucleic acid aptamer beacons.

In order to achieve the above object, the present disclosure, in one aspect, includes the following inventions:

### Invention 1

A method for detecting a target substance using a nucleic acid aptamer beacon,
the nucleic acid aptamer beacon comprising:
(1) a region of a first substance that emits fluorescence,
(2) a region of a second substance that modulates light emission by the first substance,
(3) a region of a first nucleic acid linked directly or indirectly to the first substance,
(4) a region of a second nucleic acid linked directly or indirectly to the second substance, and
(5) a target substance-binding region configured to bind to the target substance and cause a conformational change in the nucleic acid aptamer beacon,
wherein the region of the first nucleic acid and the region of the second nucleic acid may be integrated or may be present separately,
the region of the first nucleic acid and the region of the second nucleic acid each comprise at least one region of a partial sequence hybridizable to each other,
wherein the method comprises:
(A) a step of mixing the target substance, the nucleic acid aptamer beacon, and a response-enhancing nucleic acid; and
(B) a step of measuring light emission from the mixture, and
wherein the response-enhancing nucleic acid comprises a region hybridizable to a portion of at least one of the region of the first nucleic acid and the region of the second nucleic acid.

### Invention 2

The method of Invention 1, wherein the first substance and the second substance are a combination in which donor fluorescence is quenched via FRET.

### Invention 3

The method of Invention 1, wherein the first substance and the second substance are a combination that induces fluorescence emission at an acceptor via FRET.

### Invention 4

The method of any one of Inventions 1 to 3, wherein the nucleic acid is DNA.

### Invention 5

The method of any one of Inventions 1 to 3, wherein the nucleic acid is RNA.

### Invention 6

The method of any one of Inventions 1 to 5, wherein the response-enhancing nucleic acid has a base length of 8 bases or more.

In one aspect, the methods of the present disclosure use a response-enhancing nucleic acid. This enhances light emission intensity and sensitivity.

### BRIEF DESCRIPTION OF THE DRAWINGS

FIG. 1 illustrates the principle of a nucleic acid aptamer beacon according to an embodiment;

FIG. 2 illustrates the principle of a nucleic acid aptamer beacon and a response-enhancing nucleic acid according to an embodiment;

FIGS. 3A-3C illustrate an effect of oligo DNA base length on fluorescence response of a DNA aptamer beacon; Oligo DNAs having different lengths were added to an adenosine assay system to compare the response of the DNA aptamer beacon to adenosine; Oligo DNA-1 (FIG. 3A), Oligo DNA-2 (FIG. 3B), and Oligo DNA-3 (FIG. 3C) were added at final concentrations of 0, 5, 20, or 80 nM; Data are presented as mean±standard deviation (n=3); and

FIG. 4A-4C illustrate an effect of an oligo DNA concentration on a change in fluorescence intensity of the DNA aptamer beacon over time; Changes in fluorescence intensity over time in an adenosine assay system when Oligo DNA-1 was added at 0 nM (FIG. 4A), 20 nM (FIG. 4B), and 80 nM (FIG. 4C) are shown; Data are presented as means (n=3);

FIGS. 5A-5C illustrate a calibration curve and Hanes-Woolf plot for adenosine; (FIG. 5A) Calibration curve for adenosine with 0, 20, or 80 nM of Oligo DNA-1 added; Data are presented as mean±standard deviation (n=3); (FIG. 5B) A regression line was created using data points in a concentration range (5-50 μM) that were above the lower limit of quantification and had a coefficient of determination (R²) of 0.99 or more on a Hanes-Woolf plot, from the data used to create (FIG. 5A).

### DETAILED DESCRIPTION OF THE INVENTION

Specific embodiments for carrying out the present disclosure will be described in detail below. The following descriptions are intended to facilitate understanding of the invention. That is, it is not intended to limit the scope of the present disclosure.

### 1. Overview

In an embodiment, the present disclosure relates to a method for detecting a target substance using a nucleic acid aptamer beacon. The method includes the following steps:
(A) a step of mixing a target substance, a nucleic acid aptamer beacon, and a response-enhancing nucleic acid; and
(B) a step of measuring light emission from the mixture.

Here, the response-enhancing nucleic acid includes a region hybridizable to a portion of at least one of the region of the first nucleic acid and the region of the second nucleic acid of the nucleic acid aptamer beacon.

In the following sections, each step and each element necessary to carry out the step will be described in detail.

### 2. Nucleic Acid Aptamer Beacon

The above method uses a nucleic acid aptamer beacon. Functionally, the nucleic acid aptamer beacon has a function of causing a conformational change in the nucleic acid when it binds to the target substance and presenting the change in a form that is easily recognized.

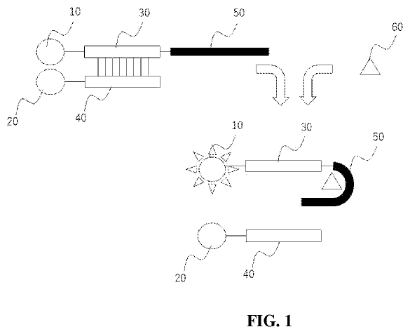

In one embodiment, the nucleic acid aptamer beacon has the following moieties (see FIG. 1):
First substance (10)
Second substance (20)
Region (30) of the first nucleic acid
Region (40) of the second nucleic acid
Target substance-binding region (50)

The first substance (10) has a function of emitting fluorescence. Here, the emitting of fluorescence refers to the absorption of light at one wavelength and the emission of light at another wavelength. The first substance (10) may also function as a donor molecule in terms of a phenomenon called FRET (Fluorescence Resonance Energy Transfer).

The second substance (20) has a function of modulating the light emission by the first substance (10). For example, the second substance (20) may function as a quencher (also referred to herein as a quenching agent). Specifically, the second substance (20) may absorb light emission at a specific wavelength by the first substance (10) and may not emit light at that wavelength or another wavelength. This allows the light emission by the first substance (10) to be quenched by the second substance (20).

The second substance (20) may also function as an acceptor molecule in terms of a phenomenon called FRET (Fluorescence Resonance Energy Transfer). Specifically, the second substance (20) can absorb light emission at a specific wavelength by the first substance (10) and emit light having another wavelength.

The region of the first nucleic acid (30) is a region of nucleic acid linked directly or indirectly to the first substance (10). The region of the second nucleic acid (40) is a region of nucleic acid linked directly or indirectly to the second substance (20).

In one example, “directly linked” may mean, for example, that the first substance (10), which is a substance other than a nucleic acid, is linked to the region of the first nucleic acid (30), which is a nucleic acid, without intervening another atom or molecule. The same is true for the second substance (20).

In another example, “indirectly linked” may mean, for example, that the first substance (10), which is a substance other than a nucleic acid, is linked to the region (30) of the first nucleic acid, which is a nucleic acid, via another substance (e.g., another atom, another molecule, such as another nucleic acid, or another linker molecule). The same is true for the second substance (20).

The target substance-binding region (50) is a nucleic acid region that binds to the target substance (60). Preferably, it is a nucleic acid region that specifically binds to the target substance (60). By way of non-limiting example, “specifically bind” may mean that the nature of binding (e.g., binding constant, and the like) to the target substance (60) differs to the extent that it produces a statistically significant difference as compared to a control. The target substance-binding region (50) and the region of the first nucleic acid (30) may be composed of different nucleic acid sequences, or the sequences of both may overlap (in other words, a portion of the target substance-binding region (50) may form a portion of the region of the first nucleic acid (30)).

With reference to FIG. 1, how each of the above-mentioned elements contributes to the detection of the target substance (60) will be described.

In one example, the region (30) of the first nucleic acid and the region (40) of the second nucleic acid are hybridized. A distance between the first substance (10) and the second substance (20), each linked to these regions, decreases. Thus, the light emission from the first substance (10) is modulated by the second substance (20). For example, when the second substance (20) functions as a quencher, the light emission from the first substance (10) is quenched.

However, if a target substance (60) is present, such a target substance (60) binds to the target substance-binding region (50). This causes at least a partial conformational change in the nucleic acid aptamer beacon. When the conformational change occurs, the region (30) of the first nucleic acid and the region (40) of the second nucleic acid are prevented from hybridizing. This results in an increased distance between the first substance (10) and the second substance (20). Thus, the light emission from the first substance (10) is not modulated by the second substance (20).

The above descriptions are only illustrative, and various modification patterns from the structure shown in FIG. 1 are present.

### 2-1. Types of Nucleic Acids (Modification Pattern 1)

The nucleic acid used may be DNA, RNA, or a combination of both (e.g., the region (30) of the first nucleic acid is DNA and the region (40) of the second nucleic acid is RNA, or the region (30) of the first nucleic acid is RNA and the region (40) of the second nucleic acid is DNA).

### 2-2. Function of First and Second Substances (Modification Pattern 2)

In one example, the first substance (10) and the second substance (20) may be a combination in which donor fluorescence is quenched via FRET. Here, quenching also includes events where fluorescence is partially attenuated. For example, the first substance (10) has a function of emitting light at a certain wavelength. The second substance (20) then has a function of absorbing light at or near that wavelength (and the second substance (20) does not have a function of emitting light).

In another example, the first substance (10) and the second substance (20) may be a combination that induces fluorescence emission at an acceptor via FRET (Fluorescence Resonance Energy Transfer). Here, the first substance (10) may function as a donor molecule and the second substance (20) may function as an acceptor molecule. For example, the first substance (10) has a function of emitting light at a certain wavelength. The second substance (20) then has a function of absorbing light at or near that wavelength. The second substance (20) can also use light at or near that wavelength as excitation light and has a function of emitting light at a different wavelength.

As another example, the first substance (10) and the second substance (20) may serve as a donor and an acceptor, respectively, in PET (Photoinduced Electron Transfer).

### 2-3. Region of First Nucleic Acid and Region of Second Nucleic Acid (Modification Pattern 3)

The region (30) of the first nucleic acid and the region (40) of the second nucleic acid may be integrated or may be present separately. FIG. 1 is an example of the latter. When they are integrated, the region (30) of the first nucleic acid and the region (40) of the second nucleic acid may be present, for example, so as to sandwich the target substance-binding region (50) and/or another nucleic acid region. When both are at least partially hybridized, the nucleic acid aptamer beacon may have a loop structure.

### 2-4. Distance Between First Substance and Second Substance (Modification Pattern 4)

In the example in FIG. 1, when the target substance (60) is not present, the distance between the first substance (10) and the second substance (20) decreases, and when the target substance (60) is present, the distance between the first substance (10) and the second substance (20) increases. In another example, when the target substance (60) is not present, the distance between the first substance (10) and the second substance (20) may increase, and when the target substance (60) is present, the distance between the first substance (10) and the second substance (20) may decrease.

As described above, nucleic acid aptamer beacons can be modified according to various modification patterns or combinations thereof.

### 2-5. Combination of First and Second Substances

The Combination of the First Substance and the Second Substance is not particularly limited, and may be, for example, a combination capable of causing FRET. In view of homo-FRET, the combination of the first and second substances may include not only combinations of different types of substances but also combinations of the same type of substance. Typically, however, the combination of the first and second substances is a combination of different types of substances. The second substance may also include a quenching agent.

By way of non-limiting example, the following combinations are included:
CFP and YFP
GFP (including EGFP) and mCherry
mTurquoise2 and m Venus
mNeonGreen and mRuby3
Fluorescein and Tetramethylrhodamine
Fluorescein and Black Hole Quencher® 1 (BHQ-1)
IAEDANS (5-[2-Iodoacetylaminoethyl] Aminonaphthalene-1-Sulfonic Acid) and Fluorescein
EDANS (5-(2-Aminoethylamino) naphthalene-1-sulfonic acid) and Dabcyl Cy3 and Cy5
Alexa Fluor 488 and Alexa Fluor 594.

In another example, the combination may be capable of causing PET.

By way of non-limiting example, the following combinations are included:
Porphyrins and quinones
Organic dyes and amines

### 3. Target Substances

The type of target substance to be detected is not particularly limited. It may be a low molecular weight organic compound, a low molecular weight inorganic compound, or a high molecular weight organic compound.

### 4. Response-Enhancing Nucleic Acid

The response-enhancing nucleic acid has a function of hybridizing at least partially to the region of the first nucleic acid, at least partially to the region of the second nucleic acid, or at least partially to both regions (e.g., when both the region of the first nucleic acid and the region of the second nucleic acid have palindromic structures). As with the nucleic acid aptamer beacon described above, the response-enhancing nucleic acid may be DNA or RNA.

The length of the response-enhancing nucleic acid is not particularly limited. However, it is preferable to have a certain length or more, since the response-enhancing nucleic acid has a function of hybridizing and competes with hybridization between the region of the first nucleic acid and the region of the second nucleic acid. For example, the length of the response-enhancing nucleic acid may be 8 bases or more, 9 bases or more, or 10 bases or more. On the other hand, the upper limit for the length of the response-enhancing nucleic acid is not particularly limited. For example, the upper limit may be 50 bases or less, 25 bases or less, or 15 bases or less. Typically, it may be shorter than either the region of the first nucleic acid or the region of the second nucleic acid.

### 5. Detection Mechanism

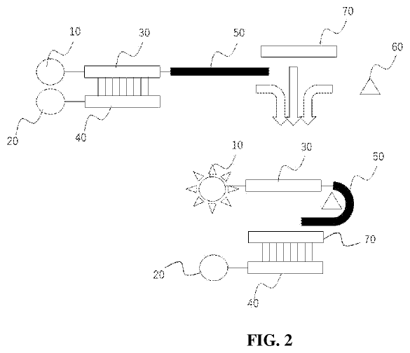

In one embodiment, the principle of detection of the target substance (60) associated with the methods according to the present disclosure is described. FIG. 2 is similar to FIG. 1, but differs from FIG. 1 in that the response-enhancing nucleic acid (70) is present.

As in FIG. 1, the region (30) of the first nucleic acid and the region (40) of the second nucleic acid are hybridized. The distance between the first substance (10) and the second substance (20), each linked to these regions, decreases. Thus, the light emission from the first substance (10) is modulated by the second substance (20). For example, when the second substance (20) functions as a quencher, the light emission from the first substance (10) is quenched.

However, if a target substance (60) is present, such a target substance (60) binds to the target substance-binding region (50). This causes at least a partial conformational change in the nucleic acid aptamer beacon. When the conformational change occurs, the region (30) of the first nucleic acid and the region (40) of the second nucleic acid are prevented from hybridizing. This results in an increased distance between the first substance (10) and the second substance (20). Thus, the light emission from the first substance (10) is not modulated by the second substance (20).

Here, the following two phenomena are in equilibrium.
(1) The target substance (60) binds to the target substance-binding region (50) and the region (40) of the second nucleic acid separates from the region (30) of the first nucleic acid.
(2) The target substance (60) separates from the target substance-binding region (50) and the region (40) of the second nucleic acid binds to the region (30) of the first nucleic acid.

When the response-enhancing nucleic acid (70) hybridizes to the region (40) of the second nucleic acid, an equilibrium shift occurs toward (1). The equilibrium shift then enhances the light emission by the first substance (10). This improves the detection sensitivity of the target substance (60).

It should be noted that in the above example, the response-enhancing nucleic acid (70) has a function of hybridizing to the region (40) of the second nucleic acid. In another example, the response-enhancing nucleic acid (70) has a function of hybridizing to the region (30) of the first nucleic acid. For example, the response-enhancing nucleic acid (70) has a function of hybridizing to a portion of the region (30) of the first nucleic acid.

As described above, when the target substance (60) binds to the target substance-binding region (50), a conformational change occurs to prevent the region (30) of the first nucleic acid from hybridizing to the region (40) of the second nucleic acid. However, if the region where hybridization is prevented is only a portion of the region (30) of the first nucleic acid, it may be possible for the response-enhancing nucleic acid (70) to hybridize to the remaining moiety. This prevents the region (40) of the second nucleic acid from hybridizing again to the region (30) of the first nucleic acid.

### 6. Other

The terms “hybridizable” and “function of hybridizing” as used herein mean that two strands of nucleic acid form a complex. Typically, the complex is formed due to the complementary relationship of the nucleic acid sequences of both strands. However, it is not essential that the nucleic acid sequences of both strands be completely complementary. For example, a complex may be formed even if only one base pair of 10 base pairs is not in a complementary relationship. Thus, the terms “hybridizable” and “function of hybridizing” not only include cases where the nucleic acid sequences of both strands are completely complementary, but also allow for non-complementarity in a small number of base pairs. For example, 10% or less (e.g., only one base pair of 10 base pairs is non-complementary), 5% or less, or 1% or less of the entire region intended to hybridize may be non-complementary.

The terms “hybridizable” and “function of hybridizing” are not limited by specific conditions (e.g., salt concentration (i.e., ionic strength), temperature, and the like). As can be understandable from the polymerase chain reaction (PCR), the two strands of nucleic acid can be separated or form a complex, depending on the temperature. In addition, the tendency of complex formation may vary depending on the concentration of inorganic salts.

However, the method of the present disclosure according to an embodiment relates to a method for detecting a target substance using a nucleic acid aptamer beacon. Therefore, it is necessary to detect the target substance under conditions where the target substance is present. Thus, the terms “hybridizable” and “function of hybridizing” may mean that the two strands may be hybridizable under conditions where the target substance is present or under conditions for detecting the target substance.

For example, in the case of detecting the presence of a product in an enzymatic reaction, if hybridization can occur at the optimal temperature (e.g., 37° C.) and optimal salt concentration for the enzymatic reaction, this falls within the concept of the terms “hybridizable” and “function of hybridizing”.

The term “nucleic acid” as used herein may or may not include naturally occurring nucleic acids. Additionally or alternatively, the term “nucleic acid” as used herein may or may not include chemically modified nucleic acids. The same is true for the terms “DNA” and “RNA” as used herein. The type of chemical modification is not particularly limited, but it may include, for example, one or more of: fluorescent labeling (e.g., FAM, TAMRA, ROX, and the like); methylation (e.g., 5-methylcytosine, methylation of the 2′-OH group of RNA, and the like); introduction of base analogs (e.g., 5-bromouracil (BrU), 6-thioguanine (6-TG), pseudouridine, and the like); fluoro-modification (e.g., 2′-fluoro-RNA (2′-F RNA)); introduction of linkers (e.g., PEGylation, biotin conjugation, and the like); polyadenylation (Poly (A) tail); morpholino nucleic acid (PMO); peptide nucleic acid (PNA); locked nucleotide (LNA); bridged nucleotide (BNA); unlocked nucleotide (UNA); glycol nucleotide (GNA); phosphorothioate bond; alkyl phosphonate diester modification; and the like.

### 7. Detection Procedure

As described above, the method according to an embodiment includes the following steps:
(A) a step of mixing the target substance, the nucleic acid aptamer beacon, and the response-enhancing nucleic acid; and
(B) a step of measuring light emission from the mixture.

Here, the step (A) may include placing the target substance, the nucleic acid aptamer beacon, and the response-enhancing nucleic acid in the same container. Furthermore, the step (A) may also include placing a precursor of the target substance, the nucleic acid aptamer beacon, and the response-enhancing nucleic acid in the same container. In this case, the precursor of the target substance is transformed into the target substance through a chemical reaction (including enzymatic reactions, and the like). The target substance produced through the chemical reaction can then be detected.

The step (B) may include, for example, irradiating the mixture with light at or near the excitation wavelength of the first substance. More specifically, the step (B) may measure the light emission from the second substance using a microplate reader or the like, or may measure the effect of quenching by the second substance.

### EXAMPLES

### Example 1 (Preparation of DNA Aptamer Beacon)

In this example, a DNA aptamer beacon (Ding and Liu, 2023) that specifically detects adenosine was used as a model. The DNA aptamer beacon was comprised of a fluorescein (FAM)-labeled aptamer strand and a quencher (Black Hole Quencher 1, BHQ1)-labeled strand (Table 1), each synthesized at Eurofins Genomics Co., Ltd. 1 μM of FAM-labeled aptamer strand, 2 μM of quencher-labeled strand, 50 mM of Tris-HCl (pH 7.5), 500 mM of NaCl, and 20 mM of MgCl₂ were mixed and heated at 95° C. for 2 minutes, and then the temperature was lowered to 25° C. over 30 minutes to hybridize both DNA strands, thereby preparing a DNA aptamer beacon solution. Three types of oligo DNAs complementary to the quencher-labeled strand and having different lengths (Oligo DNA-1, Oligo DNA-2, and Oligo DNA-3) (Table 1) were synthesized by Integrated DNA Technologies, Inc.

TABLE 1
Nucleotide Sequences and Modification Sites of Oligo DNAs Used

| Oligo DNA Name | Nucleotide Sequence and Modification Sites |
|---|---|
| FAM-labeled aptamer | 5′- |
| strand | [FAM]CTCTCGACGACGTTTGCGATGAGAAACGTATGGTTTCG |
| | A |
| | AGGTCGTC-3′ (SEQ ID NO: 1) |
| Quencher-labeled | 5′-AGTCGTCGAGAG[BHQ1]-3′ (SEQ ID NO: 2) |
| strand | |
| Oligo DNA-1 | 5′-CTCTCGACGACT-3′ (SEQ ID NO: 3) |
| Oligo DNA-2 | 5′-CTCTCGACGA-3′ (SEQ ID NO: 4) |
| Oligo DNA-3 | 5′-CTCTCGAC-3′ (SEQ ID NO: 5) |

### Example 2 (Creating Adenosine Calibration Curve Using DNA Aptamer Beacon)

Quantification of adenosine using a DNA aptamer beacon was performed in a polystyrene 96-well plate, and fluorescence measurements (Ex 480 nm, Em 530 nm) were made at 30° C. using a microplate reader (Synergy H1, BioTek). The volume of the liquid in each well was 100 μL, and the composition of the assay solution was adenosine (each concentration), DNA aptamer beacon (20 nM as FAM-labeled aptamer strand), oligo DNAs (Oligo DNA-1, Oligo DNA-2, and Oligo DNA-3) (each concentration), 50 mM of Tris-HCl (pH 7.5), 0.1 mM of MgCl₂, and 100 mM of NaCl.

### Results

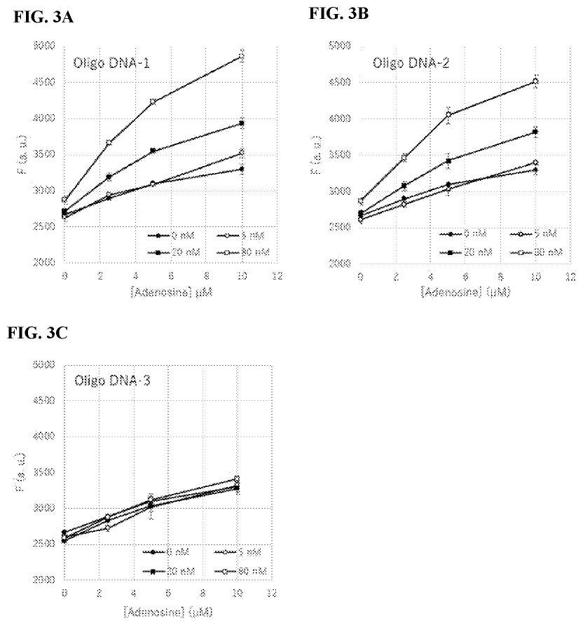

To verify the effect of suppressing the dissociated quencher-labeled strand from re-hybridizing to the FAM-labeled aptamer strand, oligo DNAs (Oligo DNA-1, Oligo DNA-2, and Oligo DNA-3), each complementary to the quencher-labeled strand, and having different lengths, were added to the assay system, and the effect on the response to adenosine was analyzed. The results showed that Oligo DNA-1 having a base length of 12-mer and Oligo DNA-2 having a base length of 10-mer significantly increased the sensitivity of the nucleic acid aptamer beacon to adenosine, with the increase in sensitivity being more effective with a longer base length and with a higher oligo DNA concentration (FIGS. 3A-3C).

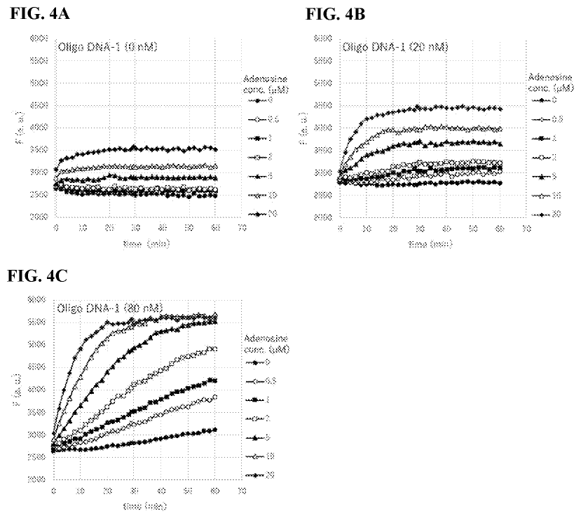

The change in fluorescence intensity over time after the addition of adenosine to the nucleic acid aptamer beacon using the oligo DNA (Oligo DNA-1) that showed the highest sensitivity-enhancing effect indicated that the rate of complex formation of adenosine and the FAM-labeled aptamer strand increased with the addition of oligo DNAs (FIGS. 4A-4C). This increase in rate was oligo DNA concentration-dependent.

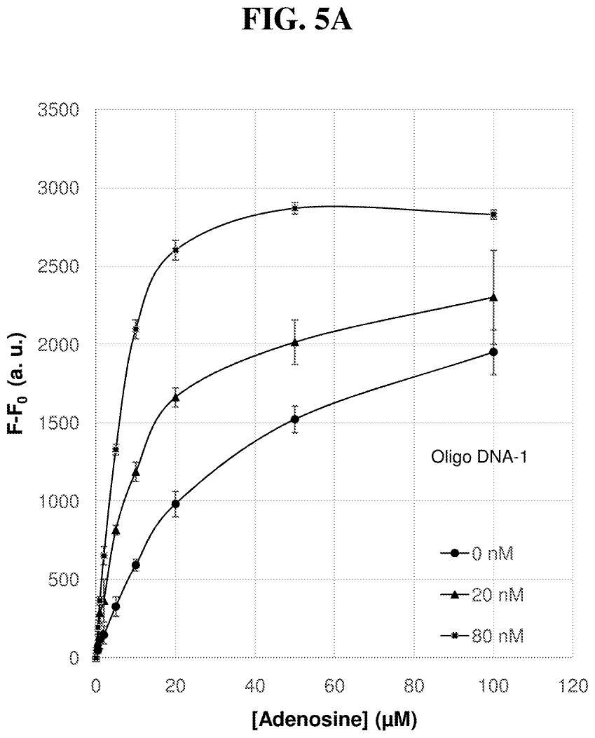

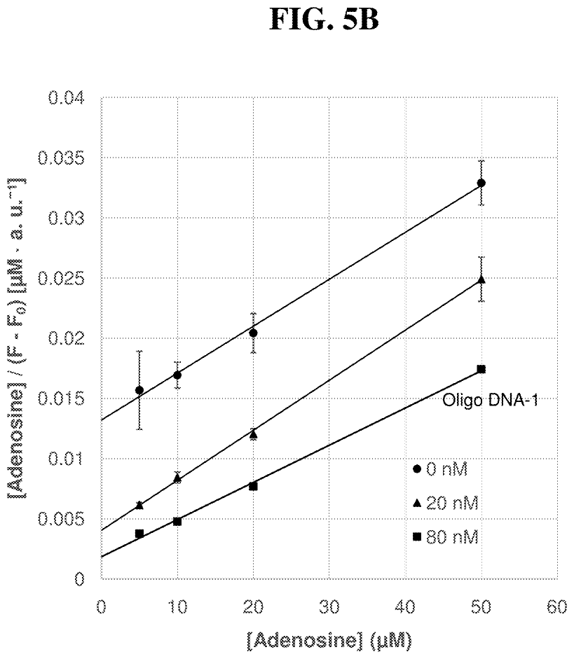

To calculate the limit of detection, limit of quantification, and apparent dissociation constant for this assay system, an adenosine calibration curve over a wider concentration range was created (FIG. 5A). The limit of detection (3.3σ/S) and the limit of quantification (10σ/S) were calculated from the standard deviation (n=10) of the fluorescence intensity (Fₒ) of the blank value (0 μM of adenosine) and the slope (S) of the linear concentration range (0-2 μM) in the calibration curve (Table 2). Apparent dissociation constants were calculated from regression lines based on Hanes-Woolf plots (FIG. 5B) created from the adenosine calibration curve data (FIG. 5A) (Table 2).

TABLE 2
Effect of Oligo DNA-1 Addition on Limit of Detection, Limit
of Quantification, and Apparent Dissociation Constant

| | Oligo DNA-1 | Oligo DNA-1 | Oligo DNA-1 |
|---|---|---|---|
| | (0 nM) | (20 nM) | (80 nM) |
| Detection Limit (μM) | 1.38 | 1.22 | 0.511 |
| Limit of Quantification | 4.19 | 3.70 | 1.55 |
| (μM) | | | |
| Apparent Dissociation | 33.8 | 9.74 | 6.00 |
| Constant (μM) | | | |

The results indicated that the addition of Oligo DNA-1 (80 nM) lowered both the limit of detection and the limit of quantification by 63%, and the apparent dissociation constant by 82%. The oligo DNA whose addition enhances the response of the nucleic acid aptamer beacon, such as Oligo DNA-1, is referred to in this example as Response Enhancing Strand (RES, which corresponds to the response-enhancing nucleic acid as described above).

Conventional nucleic acid aptamer beacons have been improved in sensitivity and specificity by introducing partial random mutations after selection by SELEX to optimize target molecule-binding sites and improve cooperativity (Zhang et al., 2017). On the other hand, the approach using the response-enhancing strand according to the present disclosure does not modify the nucleic acid aptamer itself, but designs and uses a response-enhancing strand that is hybridizable to the base sequence exposed after interaction with the target molecule, thereby suppressing the decrease in sensitivity due to re-hybridization and achieving signal enhancement. This simple and modular strategy is expected to enable efficient functional improvement because it can be easily applied to existing nucleic acid aptamer beacons at low cost without the need for complex mutagenesis as in conventional methods.

In the above examples, the following literature was consulted.

### REFERENCES

### Non-Patent Literature

Ding Y, Liu J (2023) Pushing adenosine and ATP SELEX for DNA aptamers with nanomolar affinity. J Am Chem Soc 145:7540-7547
Zhang Z, Oni O, Liu J (2017) New insights into a classic aptamer: binding sites, cooperativity and more sensitive adenosine detection. Nucleic Acids Res 45:7593-7601

The specific embodiments according to the present disclosure have been described above. The above embodiments are only specific examples, and the present disclosure is not limited to the above embodiments. For example, the technical features disclosed in one of the above embodiments can be applied to other embodiments. Unless otherwise noted, for a particular method, some steps may be interchanged with the order of other steps, and further steps may be added between two specific steps. The scope of this invention is defined by the claims.

### DESCRIPTION OF REFERENCE NUMERALS

10 region of first substance
20 region of second substance
30 region of first nucleic acid
40 region of second nucleic acid
50 target substance-binding region
60 target substance
70 response-enhancing nucleic acid

1. A method for detecting a target substance using a nucleic acid aptamer beacon,
the nucleic acid aptamer beacon comprising:
(1) a region of a first substance that emits fluorescence,
(2) a region of a second substance that modulates light emission by the first substance,
(3) a region of a first nucleic acid linked directly or indirectly to the first substance,
(4) a region of a second nucleic acid linked directly or indirectly to the second substance, and
(5) a target substance-binding region configured to bind to the target substance and cause a conformational change in the nucleic acid aptamer beacon,
wherein the region of the first nucleic acid and the region of the second nucleic acid may be integrated or may be present separately,
the region of the first nucleic acid and the region of the second nucleic acid each comprise at least one region of a partial sequence hybridizable to each other,
wherein the method comprises:
(A) a step of mixing the target substance, the nucleic acid aptamer beacon, and a response-enhancing nucleic acid; and
(B) a step of measuring light emission from the mixture,
wherein the response-enhancing nucleic acid comprises a region hybridizable to a portion of at least one of the region of the first nucleic acid and the region of the second nucleic acid.

2. The method of claim 1, wherein the first substance and the second substance are a combination in which donor fluorescence is quenched via FRET.

3. The method of claim 1, wherein the first substance and the second substance are a combination that induces fluorescence emission at an acceptor via FRET.

4. The method of claim 1, wherein the nucleic acid is DNA.

5. The method of claim 2, wherein the nucleic acid is DNA.

6. The method of claim 3, wherein the nucleic acid is DNA.

7. The method of claim 1, wherein the nucleic acid is RNA.

8. The method of claim 2, wherein the nucleic acid is RNA.

9. The method of claim 3, wherein the nucleic acid is RNA.

10. The method of claim 1, wherein the response-enhancing nucleic acid has a base length of 8 bases or more.

11. The method of claim 2, wherein the response-enhancing nucleic acid has a base length of 8 bases or more.

12. The method of claim 3, wherein the response-enhancing nucleic acid has a base length of 8 bases or more.


SEQUENCE LISTING (4 sequences)

SEQ ID NO: 1 - DNA, 47 bp, synthetic construct
  source 1..47: mol_type = other DNA
ctctcgacgacgtttgcgatgagaaacgtatggtttcgaaggtcgtc

SEQ ID NO: 2 - DNA, 12 bp, synthetic construct
  source 1..12: mol_type = other DNA
agtcgtcgagag

SEQ ID NO: 3 - DNA, 12 bp, synthetic construct
  source 1..12: mol_type = other DNA
ctctcgacgact

SEQ ID NO: 4 - DNA, 10 bp, synthetic construct
  source 1..10: mol_type = other DNA
ctctcgacga